(19) United States
(12) Patent Application Publication
(10) Pub. No.: US 2014/0128842 A1
(43) Pub. Date: May 8, 2014

(54) Medicament Administration

(71) Applicant: Robert Deberadine, Bridgewater, NJ (US)

(72) Inventor: Robert Deberadine, Bridgewater, NJ (US)

(73) Assignee: SANOFI-AVENTIS DEUTSCHLAND GMBH, Frankfurt am Main (DE)

(21) Appl. No.: 14/126,295

(22) PCT Filed: Jun. 12, 2012
(86) PCT No.: PCT/EP2012/061052

§ 371 Date: Dec. 13, 2013

(30) Foreign Application Priority Data
Jun. 14, 2011 (EP) 11169857.7

(51) Int. Cl. A61M 5/315; A61M 5/172; A61M 5/20; A61M 5/42

(52) U.S. Cl. CPC A61M 5/31571; A61M 5/427; A61M 5/172; A61M 5/20; A61M 5/422
USPC 604/506; 604/116

(57) ABSTRACT

A system comprises a pad and an injection device. A first side of the pad is configured to be attached to a skin of a user; the pad is provided with coded information; the injection device is configured to read the coded information from the pad when the injection device is brought into proximity of or contact with the pad; the injection device is configured to determine whether the coded information on the pad meets a predetermined criterion; and the injection device is configured to respond to a positive determination by autoinjecting medicament through the pad into the user, permitting medicament administration, or indicating to the user that medicament administration is appropriate. The pad and the injection device also are separate aspects.

### CROSS REFERENCE TO RELATED APPLICATIONS

The present application is a U.S. National Phase Application pursuant to 35 U.S.C. §371 of International Application No. PCT/EP2012/061052 filed Jun. 12, 2012, which claims priority to European Patent Application No. 11169857.7 filed Jun. 14, 2011. The entire disclosure contents of these applications are herewith incorporated by reference into the present application.

### FIELD OF INVENTION

This invention relates generally to medicament administration, and in particular it relates to a system comprising an injection device, to an injection device, and to a dermal pad.

### BACKGROUND

It is known for users to be able to self administer medicaments. Using hypodermic syringes, however, involves some skill and is not suitable for many users. Insulin pens are known to be used for allowing sufferers of diabetes to self inject with insulin. The generic term for such devices is injection device or injection pen, and indeed they are often used for other types of medicament including treatments for arthritis, anaemia, anaphylaxis etc. Many different types of injection pen are available on the market, and it is anticipated that some users may be confused and be unable to determine easily which of a number of injection pens available to them contains the correct medicament for a given instance. Some users also have problems administrating even if they have the correct medicament.

WO 2009/144726 discloses a portable infusion device that includes a dispensing unit to dispense therapeutic fluid, and a validation mechanism to enable operation of the dispensing unit based, at least in part, on a determination of whether operation is authorized. This document is concerned only with infusion devices and is not concerned with injection pens.

### SUMMARY

A first aspect of the invention provides a system comprising a pad and an injection device, wherein:

a first side of the pad is configured to be attached to a skin of a user;

the pad is provided with coded information;

the injection device is configured to read the coded information from the pad when the injection device is brought into proximity of or contact with the pad;

the injection device is configured to determine whether the coded information on the pad meets a predetermined criterion; and

the injection device is configured to respond to a positive determination by one of:

a) autoinjecting medicament through the pad into the user,

b) permitting medicament administration, and

c) indicating to the user that medicament administration may be appropriate.

In the system:

a well may be defined on a second side of the pad;

the injection device may have a tip that is arranged to be located in the well; and the injection device may be configured to read the coded information from the pad when the tip of the medicament administration device is located in the well.

The injection device may be configured to respond to a positive determination by permitting medicament injection by allowing operation of an activator by a user/patient.

The injection device may be configured to respond to a positive determination by autoinjecting medicament through the pad into the user.

The injection device may be configured to determine whether the coded information on the pad meets a predetermined criterion by comparing the coded information with a cartridge identifier that may be determined by the injection device.

The injection device may be configured to determine whether the coded information on the pad meets a predetermined criterion by comparing the coded information with a cartridge size adjuster setting of the injection device.

The coded information may be transferred from the pad to the injection device wirelessly.

The coded information may be transferred from the pad to the injection device through electrically conductive contacts in each of the pad and the injection device. Here, the configuration of the electrically conductive contacts provides the coded information. Alternatively, the coded information may be stored in memory forming part of the pad.

The coded information may include information identifying a medicament and the injection device may be configured to determine whether the coded information on the pad meets a predetermined criterion by identifying whether a medicament identified by the coded information received from the pad matches medicament stored within the injection device.

The coded information may include information identifying a medicament dosage and the injection device may be configured to read the information identifying the medicament dosage from the pad and to determine whether the coded information on the pad meets a predetermined criterion by determining whether dosage quantity information associated with the injection device is consistent with the read medicament dosage.

Alternatively, the coded information may include information identifying a medicament dosage and the injection device may be configured to read the information identifying the medicament dosage from the pad and to autoinject a quantity of medicament that is consistent with the read medicament dosage.

The coded information may include information identifying a permitted medicament temperature or temperature range and the injection device may be configured:

to measure a temperature of the medicament stored in the injection device;

to determine whether the coded information on the pad meets a predetermined criterion by determining whether the temperature of the medicament is consistent with the read medicament temperature information.

The medicament delivery device may comprise a controller configured to read the coded information and to control medicament delivery by autoinjection.

The first side of the pad may be provided with an adhesive. Alternatively, the pad could be attached to the skin if a user by compression, for instance provided by a strap.

The first side of the pad may be provided with an anaesthetic or pain relief medicament that is absorbable through the skin of the user.

A second aspect of the invention provides a dermal pad comprising first and second sides, wherein the first side of the pad may be configured to be secured to skin of a user and wherein the pad may be provided with coded information for reading and decoding by an injection device, the pad being pierceable by a needle of an injection device.

A well may be defined on the second side, and wherein the pad may be provided with coded information for reading and decoding by the injection device when a tip of the injection device is located in the well.

The dermal pad may comprise an RFID tag configured to store the coded information and to communicate the coded information to the injection device.

The second side of the pad may be provided with electrically conductive contacts, to allow communication of the coded information to an injection device by electrical conduction. Here, the configuration of the electrically conductive contacts provides the coded information. The coded information may alternatively be stored in memory forming part of the pad and may be communicated through the electrically conductive contacts.

The dermal pad may be affixable to the skin of a user through an adhesive layer on the pad or through the use of a strap, for instance.

A third aspect of the invention provides an injection device configured:

to read coded information from a pad when the injection device is brought into proximity to or contact with the pad;

to determine whether the coded information on the pad meets a predetermined criterion; and

to respond to a positive determination by one of:

a) autoinjecting medicament through the pad into the user,

b) permitting medicament administration, and indicating that medicament administration is appropriate.

The device may be configured to read the coded information from the pad when a tip of the injection device is located in a well defined on the pad.

The device may be configured to respond to a positive determination by permitting medicament injection by allowing operation of an activator by a user/patient.

The device may be configured to respond to a positive determination by autoinjecting medicament through the pad into the user.

The injection device may be configured to determine whether the coded information on the pad meets a predetermined criterion by comparing the coded information with a cartridge identifier that is determined by the injection device.

The injection device may be configured to determine whether the coded information on the pad meets a predetermined criterion by comparing the coded information with a cartridge size adjuster setting of the injection device.

The medicament delivery device may comprise a controller configured to read the coded information and to control medicament delivery by autoinjection.

A fourth aspect of the invention provides a method comprising:

an injection device reading coded information from a pad when the injection device is brought into proximity to or contact with the pad;

the injection device determining whether the coded information on the pad meets a predetermined criterion; and

the injection device responding to a positive determination by one of:

a) autoinjecting medicament through the pad into the user,

b) permitting medicament administration, and

c) indicating that medicament administration is appropriate.

The method may comprise the injection device reading coded information from the pad when a tip of the injection device is located in a well defined on the pad.

The method may comprise responding to a positive determination by permitting medicament injection by allowing operation of an activator by a user/patient.

The method may comprise responding to a positive determination by autoinjecting medicament through the pad into the user.

The method may comprise determining whether the coded information on the pad meets a predetermined criterion by comparing the coded information with a cartridge identifier that is determined by the injection device.

The method may comprise determining whether the coded information on the pad meets a predetermined criterion by comparing the coded information with a cartridge size adjuster setting of the injection device.

### BRIEF DESCRIPTION OF THE DRAWINGS

Embodiments will now be described, by way of example only, with reference to the accompanying drawings, in which:

FIG. 1 is a schematic perspective view of a dermal pad according to aspects of the invention;

FIG. 2 is a cross-section through the dermal pad of FIG. 1 in conjunction with an injection device, according to aspects of the invention;

FIG. 3 is an end view of the injection device shown in part in FIG. 2;

FIG. 4 is a schematic drawing illustrating components of the injection device of FIGS. 2 and 3 and there interconnection;

FIG. 5 is a flow chart illustrating operation of one embodiment of the injection device of FIGS. 2-4;

FIG. 6 is a flow chart illustrating operation of another embodiment of the injection device;

FIGS. 7 and 8 are schematic drawings illustrating components of further embodiments of injection devices according to the invention; and FIGS. 9 to 11 are flow charts illustrating operation of the injection devices of FIGS. 7 and 8.

### DETAILED DESCRIPTION

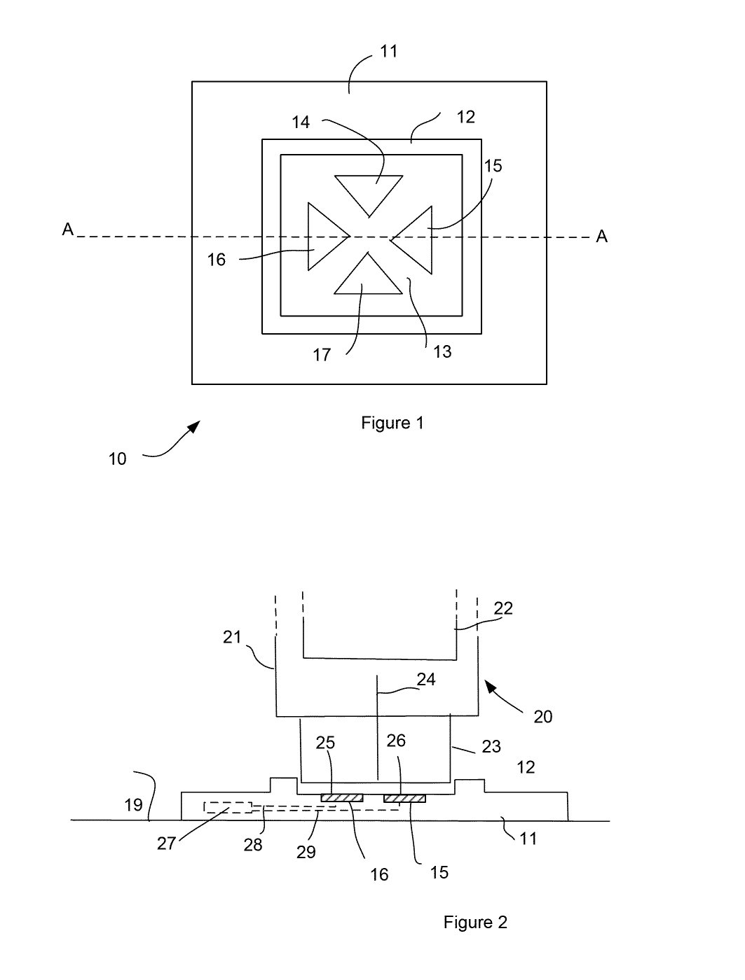

FIG. 1 illustrates a dermal pad 10 in plan view. Here the dermal pad 10 is in the form of a small patch. A cross-section through A-A of FIG. 1 is shown in FIG. 2, and the following description will make reference to both of these Figures.

The dermal pad 10 may be fixed on the skin, for example by an adhesive layer on the bottom side of the dermal pad 10. The dermal pad has a function of guiding an injection device towards the injection site. For example, the injection site may be in the center of the dermal pad. Thus, the user, for example the patient, a health care professional or a physician, can identify the position of the injection and hold an injection device at or against the indicated position.

The dermal pad 10 includes a main planar portion 11. Centrally in the pad 10 is provided a continuous raised portion 12. Here, the raised portion 12 is generally square shaped. The square raised portion 12 encircles an area 13 that is a continuation of the planar part 11. As can be seen best in FIG. 2, the raised portion 12 defines a well centrally in the patch 10.

Within the well, which will hereafter be referred to as well 13, are formed first to fourth electrical contacts 14, 15, 16, 17. In this example, they are generally triangular in shape and are directed towards the central point of the patch 11, although they may take any other suitable form. The contacts 14-17 are formed of an electrically conducting material, for instance a metal. As can be seen best in FIG. 2, the contacts 14-17 have an uppermost surface that is flush with the surface of the planar part 11. As such, the contacts 14-17 are partially buried within the material of the planar part 11.

Optionally, the patch 10 includes an information coder 27 which is connected to the contacts 14-17 by conductive tracks, two of which are illustrated at 28 and 29 in FIG. 2.

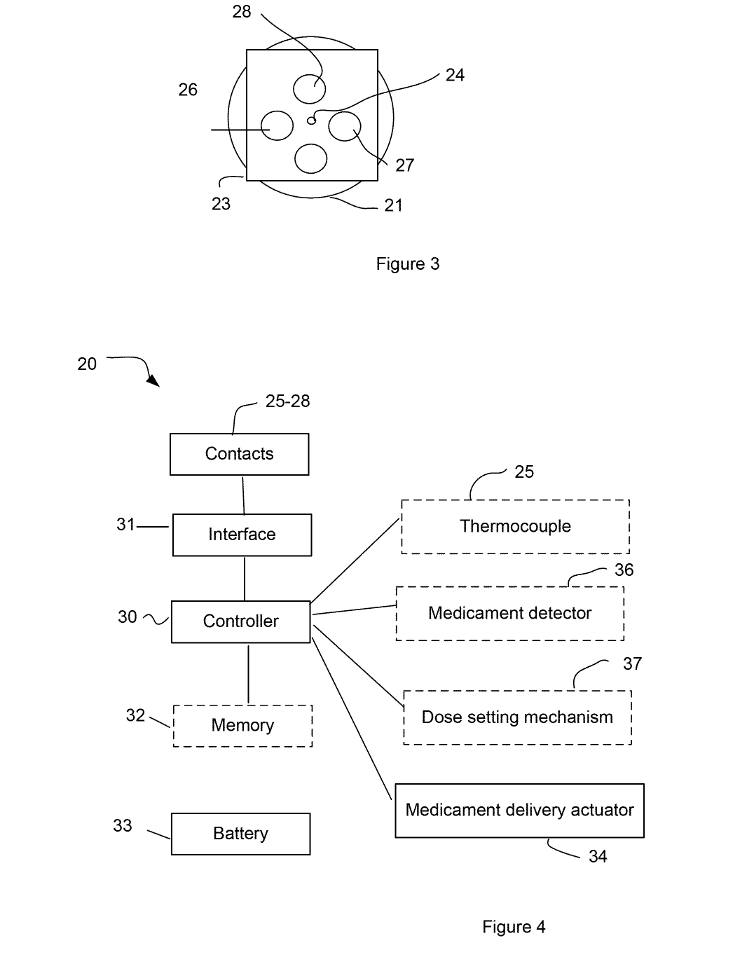

Also shown in FIG. 2 is part of an injection device 20, which in this example is an injection pen. Only some components of the injection pen 20 are shown in FIG. 2, and some others are shown in FIGS. 3 and 4.

FIG. 2 shows the injection device, or injection pen, 20 in cross-section and shows only part of the distal end of the pen 20. A part of the main body 21 is shown as containing a reservoir 22 of medicament. The reservoir may be part of a replaceable cartridge, for instance. A tip 23 is supported at the end of the body 21. The tip supports a needle 24, which is shown in FIG. 2 in a retracted position. At the distal end of tip 23 are provided first to fourth contacts 25, 26, 27 and 28. As can be seen best from FIG. 2, the contacts 25-28 protrude from the end face of the tip 23. As best seen in FIG. 3, the contacts 25-28 may be generally circular in shape. The contacts 25-28 may be tapered from a relatively wide part where the contacts are connected to the tip 23 to a relatively narrow part at their other end.

The tip 23 has a generally square shaped cross section. The square formed by the cross section of the tip 23 is slightly smaller than the internal dimension of the well 13 formed by the raised portion 12 of the pad 10. Furthermore, the height of the raised portion 12 is greater than the height of the contacts 25-28 of the tip 23. The raised portion 12 and the tip 23 thus cooperate to allow a user to cause the tip 23 to be located within the well 12 at a particular orientation, or more particularly at one of four particular orientations. Alternative configurations for the shape of the raised portion 12 and the shape of the tip 23 will be envisaged by the skilled person.

When the tip 23 is located in the well formed by the raised portion 12 of the pad 10, the contacts 25-28 of the tip 23 connect mechanically and electrically with the contacts 14-17 of the pad 10. This allows the injection pen 20 to derive information from the pad 10. There are a number of possible alternatives for this, some of which will now be explained.

In a simple embodiment, the pad 10 provides information in the connection of different ones of the contacts 14-17 to other contacts. The connection of a contact 14-17 of the pad 10 to other contacts on the pad 10 can be detected by the injection pen 20 in a number of ways, as described below.

For completeness, some different permutations will now be described. In a first permutation, two adjacent contacts are electrically connected to each other and the other two contacts are electrically isolated from all of the other contacts. In a second permutation, opposite contacts are electrically connected to one another, and the two other contacts are electrically isolated from all of the other contacts. In a third permutation, one contact is electrically connected to the opposite contact but is isolated from the other contacts, which are electrically connected to one another. In a fourth permutation, adjacent contacts are connected to one another and are electrically isolated from the other contacts, which are electrically connected to each other. It will be appreciated that there are further permutations that can resolve from this arrangement and that produce unique results when analysed by the injection pen 20.

As mentioned above, the connection of a contact 14-17 of the pad 10 to other contacts on the pad 10 can be detected by the injection pen 20 in a number of ways. For instance, the injection pen 20 may cause an electrical signal to be provided at one of the contacts 25-28 of the injection pen 20 and simultaneously detect whether the signal is present on the other ones of the contacts. If there is a direct electrical connection between two of the contacts 14-17 on the pad 10, an electrical circuit will be provided between two of the pads 25-28 on the injection pen 20. As such, detecting which of the first to third contacts 25-27 of the injection pen 20 are connected to the fourth contact 28 when the fourth contact 28 is energised allows the injection pen to determine which of the contacts 14-17 of the pad 10 are electrically connected to the contact that is coupled directly to the fourth contact 28 of the injection pen 20. By performing the same process for electrical signals applied to each of the contacts 25-28 of the injection pen 20 in turn (although one can be omitted), the injection pen 20 can determine with certainty how the contacts 14-17 of the pad 10 are connected to one another. Because the orientation of the tip 23 of the injection pen 20 in the well 13 formed by the raised portion 12 of the pad 10 is not known, the number of permutations of electrical connections of the pads 14-17 is lower than the number of permutations that will be present if the orientation was known. However, this arrangement can indicate a significant amount of information because there is a significant number of different permutations of connections of the contacts 14-17 of the pad 10.

In the above, the information coder 27 merely includes fixed electrical connections between the various contacts 14-17.

In alternative embodiments, the information coder 27 is more sophisticated. For instance, the information coder 27 may include an active electrical circuit comprising one or more transistors. The information coder 27 may be responsive to being provided with electrical power to communicate information to the injection pen 20 through the contacts 14-17 of the pad 10. Communication may be effected in any suitable way, for instance as a serial data signal provided on one or more electrical connections made through the contacts 14-17. The information coder 27 may include a microprocessor or microcontroller that is responsive to a query provided by the injection pen 20 through the contacts of the injection pen and the contacts 14-17 of the patch 10 to respond with coded information, which may be communicated in any suitable way.

In an embodiment, the injection pen 20 and the pad 10 communicate wirelessly. Thus, the contacts 25-28 and 14-17 may not be needed, or may only be used to supply power to the pad 10. Alternatively, the pad 10 comprises a battery.

In some example embodiments, however, power is supplied to the pad by a coil in the tip 23 and received by the pad 1 through a second coil, for example in raised portion 12. Thus, power and information can be exchanged wirelessly through an electro-magnetic field in a manner similar to that used in RFID tags.

The information provided on the pad 10 comprises information regarding a particular medicament. The information may additionally indicate a medicament dose. Information on the medicament and the dose may be especially assigned to the user or patient of the pad 10. The information may additionally indicate a medicament temperature or medicament temperature range. The way in which the medicament, the dose and the temperature are indicated in the information is not important, and various alternatives will be envisaged by the skilled person. The information may be programmed into the pad 10 for example through contacts 14-17 or wirelessly. The information may be stored in a memory, for example a FLASH memory, or a programmable read-only memory (PROM).

The pad may be designed for single use or for repeated use. For example, the pad needs to be replaced when the injection site on the patient's body needs to be changed. In some embodiments, the pad is able completely to be removed from the body and disposed. In alternative embodiments, the adhesive layer can be removed from the pad and be replaced. Here, the mechanic and electronic parts may be reused with a new adhesive layer. In further embodiments, the pad 10 may be fixed to a patient, e.g. to an arm, with a strap. Thus, the user could change the location of a subsequent injection with the same pad.

FIG. 4 shows some components of the injection pen 20.

The contacts 25-28 are shown as being connected to a controller 30 by an interface 31. The controller 30 may take any suitable form, for instance a microcontroller or microprocessor. A memory 32 may be connected to the controller 30, or alternatively the controller 30 may include some internal memory (not shown). Components of the injection pen 20 are powered by a battery 33.

Also coupled to the controller 30 is a medicament delivery actuator 34. The medicament delivery actuator 34 includes one or more electrical transducers, and is operable to cause injection of medicament from the reservoir 22 into a user, for example an automatic injection or autoinjection.

In FIG. 2, the pad 10 is shown as being provided on skin 19 of a user. Although not shown in the Figure, the surface of the planar part 11 of the pad 10 that is opposite to the surface on which the raised portion 12 is formed is provided with an adhesive layer, in order to allow the pad 10 to be secured to the skin 19 in an easily removable way. This surface of the planar part 11 of the pad 10 or of the adhesive layer may also be provided with an anaesthetic or pain relief medicament that can be absorbed into the skin 19. The anaesthetic may for instance be a substance containing Lidocaine.

The medicament delivery actuator 34 operates in three stages. In the first stage, the needle 24 is forced in a direction that is downwards in FIG. 2 so as to pierce the pad 10 and to result in its distal end being provided in the flesh of the user below the skin layer 19. In the second stage, medicament stored in the reservoir 22 is caused to be expelled through the needle 24 into the user. In the third stage, the needle 24 is caused to be retracted from the flesh of the user into the injection pen 20. The medicament delivery actuator 34 may take any suitable form. A difference between a medicament injection device for use with a pad 10 and a medicament injection device for use without a pad is that the travel of the needle 24 is greater, in order to compensate for the additional distance that the needle 24 must travel through the planar part 11 of the pad 10 in order to achieve a given depth of injection into the user. The injection depth achieved by the length of the travel of the needle 24 may be defined by a mechanical stop in the medicament delivery actuator 34.

The injection pen 20 optionally includes a thermocouple 35 or other temperature sensing device. The thermocouple 35 is configured to detect a temperature of a medicament included in the reservoir 22 of the injection pen 20.

The injection pen 20 also optionally includes a medicament detector 36. This may take any suitable form. The medicament detector 36 is configured to detect a type of medicament that is present in the reservoir 22. The medicament detector 36 may operate by detecting some physical characteristic of the reservoir/cartridge 22 itself, for example a bar code or sigil printed on a label of the cartridge 22, a material, physical dimension, color or shape of the cartridge 22 or the like, or may operate by detecting a physical or chemical characteristic of the medicament itself.

The injection pen 20 optionally includes a dose setting mechanism 37. If a dose setting mechanism 37 is provided, the medicament delivery actuator 24 is operable to control a dose of medicament provided to the user by injection.

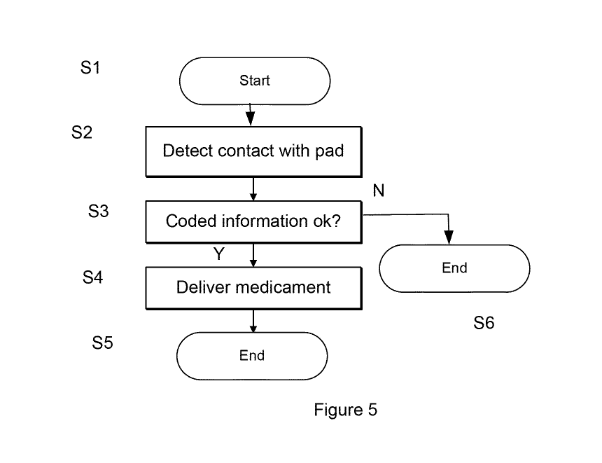

Operation of the injection pen 20 according to one embodiment will now be described with reference to FIG. 5. Here, the injection pen 20 has a relatively simple form. The controller 30 may take the form of a microcontroller, instead of a microprocessor, and the memory 32 may be omitted.

The injection pen 20 is absent of a thermocouple 35, and absent of a medicament detector 36 and absent of a dose setting mechanism 37. The injection pen 20 of this embodiment is provided with some information relating to a pad 10 with which the injection pen 20 is allowed to be used. This information may take a relatively simple form, for instance a few bits of information.

The operation of FIG. 5 starts at step S1. At step S2, the injection pen detects contact with the pad 10. This may occur in any suitable way. Contact between the pen 20 and something, for instance the pad 10, may be detected for instance by a mechanical switch. Alternatively, it may be detected by detecting an electrical connection between two of the contacts 25-28 of the tip 23.

Following detection of contact with the pad 10 at step S2, at step S3 the injection pen 20 determines whether coded information on the pad 10 is consistent with the information stored within the pen 20. This can be achieved in any suitable way. For instance, this may be achieved by applying an electrical signal to one of the contacts 25-28 and detecting electrical signals at the other ones of the contacts, and by then repeating for the other contacts, as described above. In this way, coded information provided by the pad 10 can be detected and compared to the information stored within the injection pen 20.

If it is determined that the coded information on the pad 10 is consistent with the information stored within the injection pen 20, medicament is delivered by injection at step S4. This step involves the controller 30 causing the medicament delivery actuator 34 to deliver medicament to the user. As mentioned above, this comprises firstly causing the needle 24 to pierce the skin 19 of the user, causing the medicament within the reservoir 22 to be dispensed through the needle 24 and then retracting the needle 24.

Following step S4, the operation ends at step S5. If it is determined at step S3 that the coded information included on the pad 10 is not consistent with the information stored within the injection device 20, the operation ends at step S6.

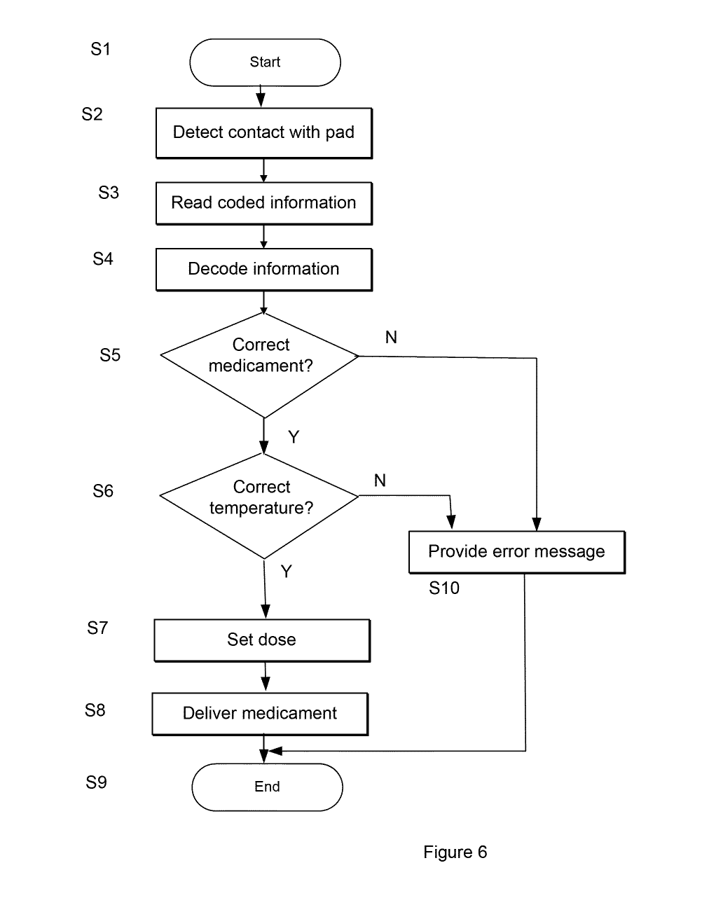

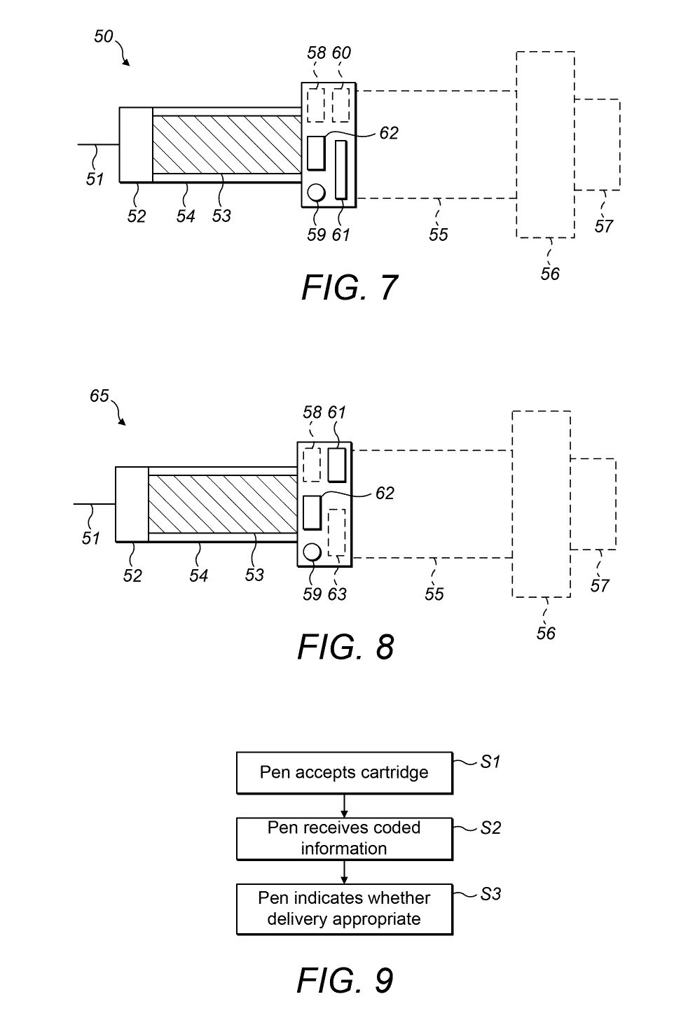

Operation of an alternative form of injection pen 20 will now be described with reference to FIG. 6. In this embodiment, the injection pen 20 may have a microcontroller or a microprocessor as the controller 30. In this embodiment, the injection pen 20 may or may not include a memory 32. The injection pen 20 here includes one or more of the thermocouple 35, the medicament detector 36 and the dose setting mechanism 37. For simplicity, this explanation assumes that all three of these features are present in the injection pen 20. However, this constitutes an explicit disclosure of possible combinations of the presence and absence of these features.

The operation starts at step S1.

At step S2, contact with the pad 10 is detected. This may occur as described above with reference to FIG. 5 or, if the pad 10 includes an active information coder 27, step S2 may involve active interrogation of the information coder 27 by the controller 30.

At step S3, the information coded onto the pad 10 is read by the controller 30. The information is decoded at step S4.

At step S5, the controller 30 determines whether the correct medicament is present in the injection pen 20. This involves comparing information forming part of the information decoded from the pad 10 and identifying a medicament to which the pad 10 relates with information identifying the medicament present in the injection pen 20. This information may be derived from the medicament detector 36, or it may be pre-programmed into the injection pen 20 upon manufacture.

On a positive determination, it is determined at step S6 whether the medicament is at the correct temperature. This step involves determining a temperature of the medicament stored in the reservoir 20 to using the thermocouple 35. The correct temperature for the medicament may be defined in terms of an absolute temperature or in terms of a range of temperatures. The correct temperature for the medicament may be provided as part of information encoded at the pad and decoded in step S4, or alternatively it may be pre-programmed into the injection pen 20 at manufacture.

At step S7, a medicament dose is set. This step involves the controller 30 identifying from the information decoded at step S4 a dose of medicament that is required to be delivered.

At step S8, the medicament is delivered by injection. The medicament is injected in any suitable way, for instance by autoinjection and/or as described above with reference to FIG. 5.

Following step S8, the operation ends at step S9.

Step S7 involves the dose setting mechanism 37 being controlled by the controller 30 to set the correct medicament dose. This may occur in any suitable way.

Following a negative determination at either at step S5 or S6, an error message is displayed at step S10 before the operation ends at step S9 without medication having been dispensed.

Because the injection pen 20 does not deliver medication until it is detected to be in place with respect to the pad, many problems sometimes found with use of injection pens can be avoided.

To cause an injection of medication, a user firstly places the pad on their skin at a location where they wish the injection to be made. Once in place, the user does not need to remain in contact with the pad because it is secured against the skin at the correct location by the adhesive. The user then takes the injection pen and manoeuvres it so that the tip 23, which at this stage has no needle projecting therefrom, is placed in the well 13 in the pad 10. Unless the well 13 and the end of the tip 23 are circular (which they are in some embodiments), this may require the user rotating the tip 23 until it is aligned with the well 13. Once the injection pen 20 is in place in the well 13, it is able to read the information coded into the injection pen. On confirming that there is correspondence between the information on the pad and information within the pen, the injection pen 20 delivers the medicament. Advantageously, this is achieved without any input from the user, other than locating the tip 23 of the injection pen in the well 13.

Features that give rise to ease of use and reduced possibility of incorrect use are particularly advantageous in the case of users that are impaired in some relevant way. In some cases, the disease that is treated by the medicament makes the user unable reliably to self-administrate the medicament. Indeed, using features of the invention, some users that previously were unable to self-administrate, or at least do so reliably, will now be able to self-administrate. The ability to self-administrate provides an improved quality of life in many patients.

In some embodiments, the pad 10 is not suitable for re-use and is discarded by the user after the injection. This provides certain advantages. In particular, the user can be provided with pads that facilitate the user to follow a predetermined medicament regime. For instance, the user can be provided with pads that are marked, either on the pad itself or on packaging containing the pad, with a time and day, day alone, or date. In this way, a user can assume that a medicament has been taken if the pad with the relevant date/time is no longer present. This can also allow a user to determine when they have forgotten to take medication, on the basis that a pad that should have been used is still present. Having this information, the user can take any needed remedial action. A unique number may be coded in the pad 10, and the pen 20 may store the information that it was used with a certain pad, for example by identifying and storing the unique number. By configuring the pen 20 such that it allows injection with a particular pad 10 only once, accidental reuse of a pad 10 can be prevented.

The provision to a user by a healthcare professional of pads specific to their medication regime can help prevention of incorrect medicament delivery to a user.

In some embodiments, the pen 20 is configured to store the unique numbers of a set of pads and be programmed with information identifying a sequence in which the pads 10 are intended to be used. For example, the pen 20 may be supplied with a set of a number (e.g. 7) numbered pads 10, which the user is required to use them in sequence. The pen 20 is configured to store information identifying which pad 10 was used last and to determine which pad 10 is permitted to be used next. Injection by the pen 20 is permitted (in any suitable way) with the pad 10 that is the next pad in the sequence.

In some embodiments, the pad 10 may be programmed with information identifying a time for a next injection or an interval between successive injections (for example once daily). In these embodiments, the pen 20 is configured to cause the user (in any suitable way) not to inject medicament outside of the permitted regime. In the case the pad 10 being programmed with information allowing a daily dose, for instance, the pen 20 may be configured not to allow injection earlier than e.g. 23 hours after the last injection.

Alternatively, the pen may be a reusable pen, and the user would get or buy a set comprising a medicament cartridge and a set of x (e.g. 7) pads. The pen might get the information on which pads to use from the cartridge. So, for example, the cartridge may have an RFID-Tag with the information on the matching pads. This is read out by the injection device, for example, when the cartridge is inserted. Thus, the reusable pen can still maintain the complete medication history for the patient.

In embodiments where the temperature of the medicament is determined to be correct before delivery is effected, this can assist in ensuring that users do not accidentally cause delivery of medicament at an inappropriate temperature.

In embodiments where dose setting is provided, the user can be prevented from administering an incorrect dose. Additionally, administration of the correct dose can be achieved without any input from the user.

Instead of the pen 20 autoinjecting the medicament, it may instead be absent of autoinjection features. Instead, the pen 20 may be configured to indicate to the user when medicament delivery is appropriate. For instance, indication may be through a transducer, such as a light source, a speaker, a vibration module etc. Alternatively or in addition, the pen 20 may be configured to allow medicament delivery only when appropriate. Some additional embodiments will now be described.

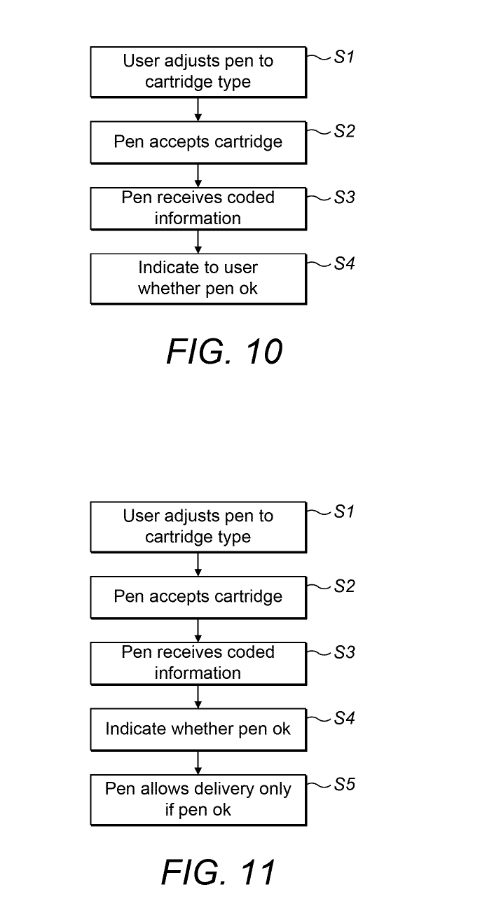

FIG. 7 is an injection device in the form of an injection pen 50. Components of the injection pen 50 include a needle 51, a pen needle support 52, a cartridge 53, a lower housing part 54, an upper housing part 55, a dose selector 56 and an activator 57.

The injection pen 50 also includes a transceiver 58, which includes a transmitter and receiver (not shown). The injection pen 50 may also include an indicator 59, which may be one or more light emitting diodes (LEDs). A cartridge detector/identifier 60 may take one of a number of different forms. In one form, it may simply detect the presence of the cartridge 53 within the injection pen 50. Alternatively, it may detect an identity of the cartridge 53. Cartridge identity detection may occur in one of a number of different ways, for example a special form or size of a cartridge, a mechanical identification, an opto-electronic identification, such as through a bar-code reader, an electronic identification, such as an RF-ID tag and reader, and/or the like.

A controller 62 is coupled to the transceiver 58, the LED 59 and the cartridge detector/identifier 60.

The injection pen 50 optionally includes a cartridge size adjuster 61. This is operable by the user/patient to set a cartridge size. The cartridge size set using the cartridge size adjuster 61 determines the size of cartridge 53 that can be inserted into the injection pen 50. The cartridge size adjuster 61 may include a screw thread mechanism for instance.

To include a new cartridge 53 into the injection pen 50, a user may separate the lower housing part 54 from the upper housing part 55. This may be performed by an unscrewing movement, utilising a screw thread mating between the two housing parts 54, 55. When the upper housing part 55 is removed, the components 58 to 62 remain coupled to the lower housing part 54 and provide an aperture for insertion of the cartridge 53. Adjustment of the cartridge size adjuster 61 by the user/patient adjusts the size and/or shape of the aperture, and thus permits only certain cartridges to be inserted into the lower housing part 54. The cartridge size adjuster 51 may be configured such as to impose a maximum diameter on cartridges that may be inserted into the lower housing part 52, in which case all cartridges of a smaller diameter can be inserted. Alternatively, the cartridge size adjuster 61 may be configured so as to allow only cartridges of a size that is set by the user/patient to be inserted into the lower housing part 54, with cartridges of other sizes (both larger and smaller diameter) being prevented from being inserted into the lower housing part 54.

The controller 62 is able to communicate with the pad 10 through the transceiver 58 and a transceiver (not shown) in the pad 10. Communication may be unidirectional, from the pad 10 to the pen 50. Alternatively, communication may be bi-directional, i.e. occur in both directions between the pad 10 and the pen 50, as discussed above. The controller 62 is configured to derive information from the cartridge/identifier. This information may simply indicate whether or not the cartridge is present, which may be detected mechanically, optically, electrically etc. Alternatively, the information may communicate the cartridge type. This may be detected by the controller 62 through detection of some identifying characteristic of the cartridge 53 itself. This may take the form of a barcode, a sigil, a conductive pattern, etc., or some physical characteristic such as size, opacity, etc. Alternatively, the information may be derived from the setting of the cartridge size adjuster 61 since the size of the cartridge 53 is dependent upon this setting.

The pen 50 and the pad 10 may be able to communicate through any suitable short-range protocol, either proprietary or standardised. The maximum range of communication may be limited by the transceivers, for instance to a maximum of 10 m, 5 m, 1 m or 10 cm. The transceivers 22 and 58 may use Bluetooth™, Bluetooth™ Low Energy, RFID, IrDA™ or some other suitable protocol.

FIG. 7 illustrates two separate embodiments. In one embodiment, the cartridge size adjuster 61 is absent. In the other embodiment, the cartridge size adjuster 61 is present.

Operation of the one embodiment will now be described with reference to FIG. 9.

Referring to FIG. 9, the injection pen 50 first accepts the cartridge 53 at step S1. This involves the user separating the first lower and upper housing parts 54, 55 and inserting the cartridge 53 into the aperture thereby formed. In this embodiment, no cartridge size adjuster 61 is present. In this embodiment, the size of the cartridge is not used to determine the cartridge type, nor the contents of the cartridge. Instead, the cartridge type and/or medicament contained within the cartridge is determined by the controller 62 and the cartridge detector/identifier 60 by analysing some aspect of the cartridge 52, for instance a bar code or other identifier attached thereto, or by analysing some aspect of the medicament, e.g. through the use of a pH sensor associated with the cartridge 53 and in contact with the medicament.

At step S2, the injection pen 50 receives the coded information from the pad 10. The injection pen 50, in particular the controller 62 thereof, compares the cartridge identifier to the received coded information. On the basis of the comparison, the controller 62 determines whether or not medicament delivery is appropriate. This determination is performed in any suitable way, for instance as described above.

At step S3, the injection pen 50 indicates to the user whether the injection pen 50 is okay to use, on the basis of the determination. This may occur in any suitable way. For instance, in the event of a positive determination, the controller 21 may illuminate a green LED 59. In the event of a negative determination, i.e. in determining that the injection pen 50 is not suitable for the user/patient, the controller 62 may illuminate a red LED 59, or alternatively cause the red LED to flash. If a determination cannot be made for any reason, this may be indicated to the user by the controller 21 illuminating a yellow LED 59. On seeing the yellow LED illuminated, the user would know to check the injection pen 50, particularly to ensure that the cartridge is installed properly and that the medicament otherwise is correct, and/or the time is correct and/or the temperature is correct etc.

The operation of the injection pen 50 described above may result in the user being presented with a warning to prevent them from administering medicaments other than in accordance with the coded information that is received from the pen 10.

Another embodiment will now be described with reference to FIGS. 7 and 10. In this embodiment, the cartridge size adjuster 61 is present in the injection pen 50.

At step S1, the user adjusts the injection pen to accept the desired cartridge size. This is achieved by the user manipulating the cartridge size adjuster 61 until a desired value or other indicator is visible at a relevant part of the exterior of the injection pen 50. The indicator may be a number, for instance “5”, or alternatively may name a medicament, for instance “insulin”. The user then proceeds to load the cartridge 53 into the lower housing part 54, in the same way as described above. Assuming the cartridge 53 is of the correct size, the injection pen 50 receives the cartridge at step S2.

The injection pen 50, and in particular the controller 62 thereof, then detects the type or identifier that is associated with the cartridge. This may occur as described above in relation to the one embodiment. Alternatively this may occur by detecting the setting of the cartridge size adjuster 61. At step S3, the injection pen 50 receives the coded information from the pad 10. At step S4, the injection pen 50, in particular the controller 62 thereof, compares the cartridge identifier to the received coded information. On the basis of the comparison, the controller 62 determines whether or not medicament delivery is appropriate. This determination is performed in any suitable way, for instance as described above. The pen 50 then indicates to the user whether or not the injection pen 50 is okay for use by the user/patient.

The third embodiment will now be described with reference to FIG. 8 and the flow chart of FIG. 11.

FIG. 8 shows an injection pen 65. Reference numerals are retained from FIG. 7 for like elements. The pen 65 also includes a delivery enabler/preventer 63.

At step S1 of FIG. 11, the user/patient adjusts the cartridge size adjuster 61 of the insulin pen 50 to the required cartridge type. This is performed as discussed above in relation to the another embodiment. At step S2, the insulin pen 50 accepts the cartridge 53. Again this is as described in relation to the one embodiment and the another embodiment. At step S3, the injection pen 50 receives the coded information from the pad 10. At step S4, the injection pen 50, in particular the controller 62 thereof, compares the cartridge identifier to the received coded information. On the basis of the comparison, the controller 62 determines whether or not medicament delivery is appropriate. This determination is performed in any suitable way, for instance as described above. The user is provided with an indication as to whether the injection pen 65 is suitable for use by the user/patient. This may occur by controlling the LED or LEDs 59 as described above. This information is also used by the injection pen 65 to control whether or not the medicament is delivered to the user/patient, as is described below. At step S4, the controller 62 may cause the LED 59 to flash quickly if the injection pen 65 is indicated as not being suitable by the user/patient and to be illuminated constantly or/flash slowly if the injection pen 65 is suitable for use by the user/patient.

At step S5, the injection pen 65 allows delivery of the contents of the cartridge 53 only if the information received from the pad 10 indicates that the cartridge 53, and thus the contents thereof, are suitable for use by the user/patient in accordance with the medicament regimen stored in the memory 25. This is effected by the delivery enabler/preventer 63 either preventing or allowing operation of the activator 57 by the user/patient to result in the injection of the contents of the cartridge 53 through the needle 51. The enabler/preventer 63 may allow delivery of the medicament in accordance with the information received from the pad 10. For example, the enabler/preventer 63 may prevent selection of a dose if a cartridge containing a wrong medicament is inserted. Alternatively or in addition, the enabler/preventer 63 may prevent selection of a dose outside a dosage window (for example a window of 10 to 20 units) in accordance with the dosage regimen for the medicament for the user/patient, as communicated by the coded information from the pad 10, but allow selection of a dose within the dosage window. Further, the enabler/preventer 63 may enable selection of a dose only after a certain time since the last administration of a dose of the medicament for example after at least 20 hours, and prevent selection of a dose otherwise. Enabling or preventing administration of a dose may occur in any suitable way.

In some other embodiments, the pen 20 is a reusable pen into which cartridges of medicament can be inserted. In these embodiments, the user may be provided with a set comprising a medicament cartridge and a set of a number (e.g. 7) of pads. The pen 20 here obtains information on which pads 10 are permitted to be used from the cartridge. For example, the cartridge may be provided with an RFID transponder or tag with the information that is described above as being stored within the pen 20 in those embodiments. This information is read out by the injection pen 20, for example, when the cartridge is first inserted into the pen 20, and the pen thereafter operates as any of the pens 20 described above. In these embodiments, the reusable pen 20 can be configured to maintain the complete medication history for the patient.

The term “medicament”, as used herein, means a pharmaceutical formulation containing at least one pharmaceutically active compound, wherein in one embodiment the pharmaceutically active compound has a molecular weight up to 1500 Da and/or is a peptide, a proteine, a polysaccharide, a vaccine, a DNA, a RNA, a antibody, an enzyme, an antibody, a hormone or an oligonucleotide, or a mixture of the above-mentioned pharmaceutically active compound, wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis, wherein in a further embodiment the pharmaceutically active compound comprises at least one peptide for the treatment and/or prophylaxis of diabetes mellitus or complications associated with diabetes mellitus such as diabetic retinopathy, wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta-decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the aforementioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be appreciated that the above-described embodiments are purely illustrative and that other embodiments will be conceived by the skilled person.

In one such embodiment, the pad 10 is configured to transfer information to a doctor (or other health care professional) in a hospital or other surgical environment, giving information on medication or a planned operation. Thus, it could be avoided that the physician gives the wrong treatment or performs the wrong operation (or e.g. on a wrong part of the body, such as accidentally operating the left knee instead of the right knee).

1-32. (canceled)

33. A system comprising a pad and an injection device, wherein:
a first side of the pad is configured to be attached to a skin of a user;
the pad is provided with coded information;
the injection device is configured to read the coded information from the pad when the injection device is brought into proximity of or contact with the pad;
the injection device is configured to determine whether the coded information on the pad meets a predetermined criterion; and
the injection device is configured to respond to a positive determination by one of:
a) autoinjecting medicament through the pad into the user,
b) permitting medicament injection, and
c) indicating to the user that medicament injection is appropriate.

34. The system of claim 33, wherein:
a well is defined on a second side of the pad;
the injection device has a tip that is arranged to be located in the well; and
the injection device is configured to read the coded information from the pad when the tip of the medicament administration device is located in the well.

35. The system of claim 33, wherein the injection device is configured to respond to a positive determination by permitting medicament injection by allowing operation of an activator by a user/patient.

36. The system of claim 33, wherein the injection device is configured to respond to a positive determination by autoinjecting medicament through the pad into the user.

37. The system of claim 33, wherein the injection device is configured to determine whether the coded information on the pad meets a predetermined criterion by comparing the coded information with a cartridge identifier that is determined by the injection device.

38. The system of claim 33, wherein the injection device is configured to determine whether the coded information on the pad meets a predetermined criterion by comparing the coded information with a cartridge size adjuster setting of the injection device.

39. The system of claim 33, wherein the coded information is transferred from the pad to the injection device wirelessly.

40. The system of claim 33, wherein the coded information is transferred from the pad to the injection device through electrically conductive contacts in each of the pad and the injection device.

41. The system of claim 40, wherein the configuration of the electrically conductive contacts provides the coded information.

42. A system as claimed in claim 33, wherein the coded information includes information identifying a medicament and wherein the injection device is configured to determine whether the coded information on the pad meets a predetermined criterion by identifying whether a medicament identified by the coded information received from the pad matches medicament stored within the injection device.

43. A system as claimed in claim 33, wherein the coded information includes information identifying a medicament dosage and wherein the injection device is configured to read the information identifying the medicament dosage from the pad and to determine whether the coded information on the pad meets a predetermined criterion by determining whether dosage quantity information associated with the injection device is consistent with the read medicament dosage.

44. A system as claimed in claim 33, wherein the coded information includes information identifying a medicament dosage and wherein the injection device is configured to read the information identifying the medicament dosage from the pad and to autoinject a quantity of medicament that is consistent with the read medicament dosage.

45. A system as claimed in claim 33, wherein the coded information includes information identifying a permitted medicament temperature or temperature range and wherein the injection device is configured:
to measure a temperature of the medicament stored in the injection device;
to determine whether the coded information on the pad meets a predetermined criterion by determining whether the temperature of the medicament is consistent with the read medicament temperature information.

46. A system as claimed in claim 33, wherein the first side of the pad is provided with an adhesive.

47. A system as claimed in claim 33, wherein the first side of the pad is provided with an anaesthetic or pain relief medicament that is absorbable through the skin of the user.

48. A dermal pad comprising first and second sides, wherein the first side of the pad is configured to be secured to skin of a user and wherein the pad is provided with coded information for reading and decoding by an injection device, the pad being pierceable by a needle of an injection device.

49. A dermal pad as claimed in claim 48, wherein a well is defined on the second side, and wherein the pad is provided with coded information for reading and decoding by the injection device when a tip of the injection device is located in the well.

50. The dermal pad of claim 48, comprising an RFID tag configured to store the coded information and to communicate the coded information to the injection device.

51. A dermal pad as claimed in claim 48, wherein the second side of the pad is provided with electrically conductive contacts, to allow communication of the coded information to an injection device by electrical conduction.

52. A dermal pad as claimed in claim 51, wherein the configuration of the electrically conductive contacts provides the coded information.

53. An injection device configured:
to read coded information from a pad when the injection device is brought into proximity to or contact with the pad;
to determine whether the coded information on the pad meets a predetermined criterion; and
to respond to a positive determination by one of:
a) autoinjecting medicament through the pad into the user,
b) permitting medicament administration, and
c) indicating that medicament administration is appropriate.

54. The device of claim 53, configured to read the coded information from the pad when a tip of the injection device is located in a well defined on the pad.

55. The device of claim 53, configured to respond to a positive determination by permitting medicament injection by allowing operation of an activator by a user/patient.

56. The device of claim 53, configured to respond to a positive determination by autoinjecting medicament through the pad into the user.

57. The device of claim 53, wherein the injection device is configured to determine whether the coded information on the pad meets a predetermined criterion by comparing the coded information with a cartridge identifier that is determined by the injection device.

58. The device of claim 53, wherein the injection device is configured to determine whether the coded information on the pad meets a predetermined criterion by comparing the coded information with a cartridge size adjuster setting of the injection device.

59. A method comprising:
an injection device reading coded information from a pad when the injection device is brought into proximity to or contact with the pad;
the injection device determining whether the coded information on the pad meets a predetermined criterion; and
the injection device responding to a positive determination by one of:
a) autoinjecting medicament through the pad into the user,
b) permitting medicament administration, and
c) indicating that medicament administration is appropriate.

60. A method as claimed in claim 59, comprising the injection device reading coded information from the pad when a tip of the injection device is located in a well defined on the pad.

61. A method as claimed in claim 59, comprising responding to a positive determination by permitting medicament injection by allowing operation of an activator by a user/patient.

62. A method as claimed in claim 59, comprising responding to a positive determination by autoinjecting medicament through the pad into the user.

63. A method as claimed in claim 59, comprising determining whether the coded information on the pad meets a predetermined criterion by comparing the coded information with a cartridge identifier that is determined by the injection device.

64. A method as claimed in claim 59, comprising determining whether the coded information on the pad meets a predetermined criterion by comparing the coded information with a cartridge size adjuster setting of the injection device.
